(19) United States
(12) Patent Application Publication
(10) Pub. No.: US 2020/0009017 A1
(43) Pub. Date: Jan. 9, 2020

(54) MULTI-VOLUME DRUG DELIVERY SYSTEM WITH VACUUM-ASSISTED MIXING AND/OR DELIVERY

(71) Applicant: Vivek K. SHARMA, San Ramon, CA (US)

(72) Inventor: Vivek K. SHARMA, San Ramon, CA (US)

(21) Appl. No.: 16/504,325

(22) Filed: Jul. 7, 2019

Related U.S. Application Data
(60) Provisional application No. 62/695,621, filed on Jul. 9, 2018.

(51) Int. Cl. A61J 1/20; A61M 5/19; A61M 39/14; A61M 39/24

(52) U.S. Cl. CPC A61J 1/2072; A61J 1/2037; A61M 39/24; A61J 1/2006; A61M 39/14; A61M 5/19

(57) ABSTRACT

A multi-chamber drug delivery system includes vacuum chambers to provided vacuum-assisted mixing of first and second desired substances and/or injection of desired substances into a target tissue of a patient.

### CROSS REFERENCE TO RELATED APPLICATIONS

This application claims the priority benefit of U.S. Provisional Application Ser. No. 62/695,621 filed Jul. 9, 2018, which is hereby incorporated herein by reference in its entirety.

### BACKGROUND OF THE INVENTION

The present disclosure relates to drug delivery systems, and more specifically to multi-chamber drug delivery systems incorporating one or more vacuum-sealed chambers to enable one or more of the storage, mixing, and injection of one or more desired substances such as a biological or pharmacological agent.

Many commercially available drug delivery systems include a mixing device or mechanism to mix one or more desired substances (e.g., a pharmacological agent and a carrier) prior to the injection of the desired substances (e.g., into a target body tissue of a patient). Such devices facilitate, for example, the mixing of a carrier or diluent with a lyophilized or powdered active substance (e.g., a drug) immediately prior to injection. Because many active substances degrade or suffer reduced activity when stored hydrated or mixed with a carrier, delivery devices which keep the two elements separate until right before administration are often used.

Drug delivery systems as described above may be provided in a variety of forms, including sequential chamber or concentric chamber designs. Sequential chamber designs involve multiple chambers in series. In a two-chamber sequence, a first chamber proximal to the device user, which is separated by a seal from a second chamber distal to the first chamber. The first chamber stores, for example, one of an active agent and a diluent, and the second chamber stores the other of the substance. Concentric barrel drug delivery systems utilize chambers that are generally coaxial with one another. In some instances, rotation of a seal is required to align a passage in the seal with an aperture or hole between a first and a second chamber to allow a carrier to be mixed with an active agent.

Because of their complexity, both sequential and concentric barrel dual chamber drug delivery systems may be expensive and/or difficult to manufacture and use. For example, some designs require the use of springs and other active members that add to the manufacturing cost and operational complexity, and reduce the reliability of the overall delivery system. In many instances, operation of dual chamber drug delivery systems is relatively complex, and untrained or poorly trained users may be unable to effectively use the device. Because the systems involve multiple chambers, complex parts, and manipulation by a user, many such device designs are also relatively bulky compared to simple single-use syringes that do not involve mixing of active agents and carriers. Further, mixing of more than two substances (e.g., two active agents and a carrier) becomes complex with these approaches.

In many cases, drug delivery systems with a syringe form factor are used to provide a highly viscous slurry or liquid solution of the active agent. In such instances, the syringe user may have difficulty mixing the carrier and active agent, because of the viscosity of one or both components or of the mixed final product. The injection process may be difficult for a user with weak hand strength to operate and may also be difficult or painful to the patient receiving the injection. In addition, there is a need to ensure that the system cannot be reused. In many instances, particularly in developing countries or remote areas, diseases are spread because syringes are reused repeatedly without adequate sterilization.

Accordingly, there is a need for improved multi-chamber drug delivery systems capable of easily and thoroughly mixing a variety of agents. There is a need for improved and simplified designs of multi-chamber drug delivery systems that are easy for untrained users to operate reliably and error-free. There is a need for multi-chamber drug delivery systems having smaller form factors and bulk compared to existing systems. There is a need for systems capable of reliably mixing high viscosity carriers and/or active agents, and which can always deliver the mixed carrier and agent without error. There is a need for drug delivery systems that may be manufactured simply, inexpensively, and using automated manufacturing processes and quality controls. There is also a need for improved drug delivery systems in which a variety of desired substances may be easily and quickly injected into a patient. Finally, there is a need for improved drug delivery systems that may be effectively employed by untrained users, such as emergency users in remote settings, and which are difficult or impossible to reuse.

### SUMMARY

In one aspect, the present invention relates to a system for delivery of at least one of a first substance and a second substance to a target body tissue of a patient, comprising: a first chamber having a first vacuum pressure and housing a first substance for delivery to a target body tissue of a patient; a second chamber having a second vacuum pressure and housing a second substance for delivery to a target body tissue of a patient; a first flow control element coupling said first chamber to said second chamber, wherein the first flow control element permits fluid communication from said first chamber to said second chamber; a first vacuum seal, wherein the first vacuum seal, when opened, exposes the first chamber to atmospheric pressure and causes the first substance to be delivered from the first chamber to the second chamber and mixed with the second substance; and a second vacuum seal, wherein the second vacuum seal, when opened, exposes the second chamber to atmospheric pressure and causes the contents of the second chamber to be delivered to the target body tissue of the patient.

In one aspect, the present invention relates to a method for delivery of at least one of a first substance and a second substance to a target body tissue of a patient, comprising: providing a first chamber having a first vacuum pressure and housing a first substance for delivery to a target tissue of a patient; providing a second chamber having a second vacuum pressure and housing a second substance for delivery to a target tissue of a patient; providing a first flow control element coupling said first chamber to said second chamber, wherein the flow control element permits fluid communication from said first chamber to said second chamber; providing a first vacuum seal that, when opened, exposes the first chamber to atmospheric pressure and causes the first substance to be delivered from the first chamber to the second chamber and mixed with the second substance; providing a second vacuum seal that, when opened, exposes the second chamber to atmospheric pressure and causes the contents of the second chamber to be delivered to the target body tissue of the patient; opening said first vacuum seal; and opening said second vacuum seal.

In one aspect, the present invention relates to a system for delivery of at least one of a first substance and a second substance to a target body tissue of a patient, comprising: a first barrel comprising: an upper first barrel chamber having a first vacuum pressure; a lower first barrel chamber having a second vacuum pressure and housing a first substance for delivery to the target body tissue of the patient; and a movable first barrel internal seal separating the upper first barrel chamber from the lower first barrel chamber; a second barrel comprising: an upper second barrel chamber having a third vacuum pressure and comprising a second vacuum seal that, when opened, exposes the upper second barrel chamber to atmospheric pressure; a lower second barrel chamber having a fourth vacuum pressure and housing a second substance for delivery to the target body tissue of the patient; and a movable second barrel internal seal separating the upper first barrel chamber from the lower first barrel chamber; a first flow control element coupling said first chamber to said second chamber, wherein the first flow control element permits fluid communication from said first chamber to said second chamber; a first vacuum seal coupled to the upper first barrel chamber that, when opened, exposes the upper first barrel chamber to atmospheric pressure and causes the first substance to be delivered from the lower first barrel chamber to the lower second barrel chamber and mixed with the second substance; and a second vacuum seal coupled to the upper second barrel chamber that, when opened, exposes the upper second barrel chamber to atmospheric pressure and causes the contents of the lower second barrel chamber to be delivered to the target body tissue of the patient.

### BRIEF DESCRIPTION OF THE DRAWINGS

FIGS. 1-3 are cross-sectional schematic views of an embodiment of a multi-chamber drug delivery device with vacuum-assisted mixing and delivery having concentric chambers.

FIGS. 4-6 are cross-sectional schematic views of an embodiment of a multi-chamber drug delivery device with vacuum-assisted mixing and delivery having parallel chambers.

FIG. 7 is a perspective view of one embodiment of the multi-chamber drug delivery device of FIGS. 4-6.

FIGS. 8-10 are cross-sectional schematic views of a multi-chamber drug delivery device with vacuum-assisted mixing and delivery having flexible bags for an active agent and a carrier.

### DESCRIPTION

Exemplary embodiments of the present disclosure are illustrated in the drawings, which are illustrative rather than restrictive. No limitation on the scope of the technology or on the claims that follow is to be implied or inferred from the examples shown in the drawings and discussed here.

In some embodiments, the invention involves multi-chamber drug delivery devices with vacuum-assisted mixing or delivery of desired substances. The devices may include two, three, four, or any desired number of chambers containing substances to be mixed immediately prior to their injection in a target body tissue of a patient. In some embodiments, the drug delivery devices are plungerless (i.e., they do not include a mechanical plunger member that drives a seal (e.g., an O-ring) to force a substance from a first volume or chamber within the system to a second volume or chamber, or to inject a mixture into a target tissue of a patient. In some embodiments, the drug delivery devices use vacuum pressures (i.e., pressures at less than atmospheric pressure) to drive a seal for mixing or injection.

As used herein, the term “vacuum” refers to a fixed or variable volume at a pressure less than atmospheric pressure. The pressure may be any pressure from a total vacuum (i.e., zero pressure) to pressures only slightly below atmospheric pressure (e.g., 0.99 atmospheres), or any pressure therebetween. As used herein, the term “chamber” refers to a fixed or variable volume within a drug delivery device having boundaries. The boundaries may be rigid, semi-rigid, or flexible.

In some embodiments, one or more chambers of the drug delivery device are maintained at a vacuum (e.g., 0.5 atm) which may be broken or unsealed by a user to cause a first substance to be delivered from a first chamber to a second chamber to mix with a second substance in the second chamber. In different embodiments, the first and second substances may comprise carriers, active agents, or both.

In some embodiments, one or more chambers of the drug delivery device are maintained at a vacuum which may be broken or unsealed by a user to cause the contents of a chamber to be injected into a target body tissue of the patient. The chamber contents may comprise a mixture created by vacuum-assisted mixing with the contents of another chamber or may comprise a substance already in its final injectable form.

In some embodiments, the invention involves methods of vacuum-assisted mixing and/or delivery of substances in a drug delivery device. In various embodiments, the methods comprise providing one or more chambers at a vacuum pressure. The one or more chambers may comprise a plurality of substances to be mixed prior to delivery to a patient, or substances in their final deliverable form. In one embodiment, the method comprises providing a first chamber having a first substance at a first vacuum, and a second chamber having a second substance at a second vacuum pressure. The method further comprises breaking the first vacuum in the first chamber to cause the first substance to be delivered to the second chamber and mixed with the second substance. In one embodiment, the method comprises breaking the second vacuum in the second chamber to cause the mixed first and second substances to be injected into a target tissue of the patient. In various embodiments, the first and second chambers may comprise sequential chambers, concentric chambers, fixed-volume chambers, or variable-volume chambers. In further embodiments, additional chambers may be provided for mixing one or more additional substances into a final deliverable mixture.

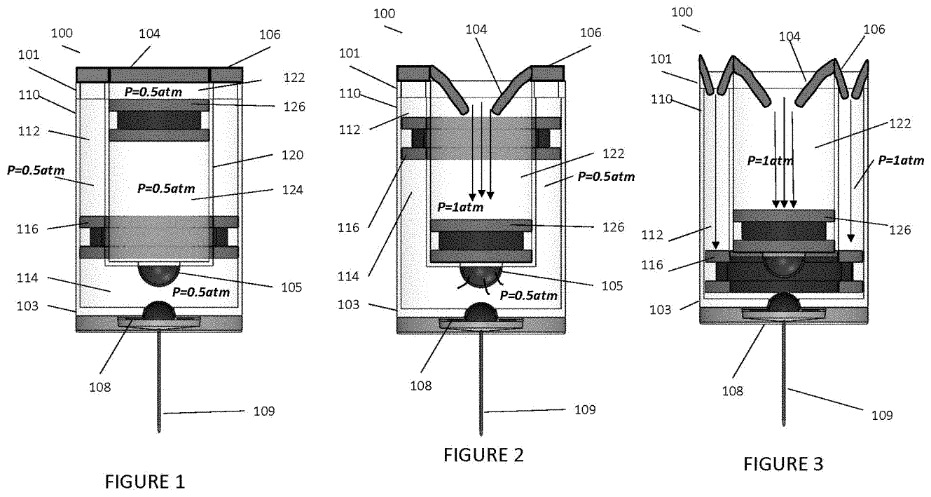

A particular embodiment of a drug delivery system according to the prevent invention is illustrated FIGS. 1-3, with like numbers referring to like elements in each figure. A cross-sectional side view of a drug delivery system 100 is illustrated in FIG. 1. The drug delivery system 100 includes a plurality of chambers having vacuum pressures to assist in the mixing of first and second desired substances within the device, and the delivery of the mixed substances to a target tissue of a patient. The drug delivery system 100 of FIG. 1 is a concentric chamber design and includes an outer barrel 110 having a first or proximal end 101 and a second or distal end 103. An inner barrel 120 is concentric with and interior to the outer barrel 110. Outer barrel 110 includes an outer barrel internal seal 116 which separates the outer barrel volume into an upper outer barrel chamber 112 and a lower outer barrel chamber 114. Outer barrel internal seal 116 is concentric with and fits engagingly around the outer periphery of inner barrel 120. Inner barrel 120 includes an inner barrel internal seal 126 that separates the inner barrel volume into an upper inner barrel chamber 122 and a lower inner barrel chamber 124. In various embodiments, the outer barrel internal seal 116 and the inner barrel internal seal 126 may be selected from an O-ring and a movable elastomeric plug.

A first substance (not shown), such as a carrier for an active agent, is provided in the lower inner barrel chamber 124. A desired vacuum pressure is provided in lower inner barrel chamber 124, which is defined by the lower surface of inner barrel internal seal 126 and the lower end of lower inner barrel 120. A first vacuum seal 104 is provided at the upper end of inner barrel 120 to seal a vacuum within the upper inner barrel chamber 122, defined by the first vacuum seal 104 and the upper surface of inner barrel internal seal 126. The vacuum pressure in upper and lower inner barrel chambers 122, 124 may be selected so as to control the force applied to inner barrel internal seal 126 when the first vacuum seal 104 is ruptured. The force is determined by the product of: a) the difference between atmospheric pressure and the pressure in lower inner barrel chamber 124, and b) the cross-sectional area of the inner barrel internal seal 126. A greater force may be selected to deliver the first substance from the lower inner barrel chamber 124 to the lower outer barrel chamber 114 more quickly, or to ensure that viscous materials may be delivered to the lower outer barrel chamber 114 within a desired time period. First vacuum seal 104 is attached to the wall defining the boundary between the inner barrel 120 and the outer barrel 110, and may comprise any of a variety of known seal types, e.g., a resilient stopper, a rupturable or puncturable membrane, etc. In preferred embodiments, first vacuum seal 104 is a seal that is easily broken by a user but is resistant to accidental breakage or rupture. In the embodiment of FIGS. 1-3, first vacuum seal 104 is a rupturable membrane seal.

FIG. 2 illustrates how first vacuum seal 104 may be ruptured to cause the first substance to be delivered from the lower inner barrel chamber 124 to the lower outer barrel chamber 114 for mixing with a second substance present in the latter chamber. When the first vacuum seal 104 is broken, entry of air into the upper inner barrel chamber 122 raises the pressure in the chamber 122 to atmospheric pressure and causes a net downward force to be applied to inner barrel internal seal 126, moving the seal 126 toward the bottom of inner barrel 120. The first substance in the lower barrel chamber is driven through a second vacuum seal 105 provided at the lower end of inner barrel 120 as the volume within lower inner barrel chamber 124 is reduced to zero, thereby causing the first substance to mix with the second substance in lower outer barrel chamber 114. The second vacuum seal 105 may be any of a variety of known seal types or other flow control elements. In the embodiment of FIGS. 1-3, the second vacuum seal 105 is a rupturable diaphragm membrane in the embodiment of FIGS. 1-3. When the inner barrel internal seal 126 reaches the lower end of inner barrel 120, the volume of lower inner barrel chamber 124 is reduced to zero (or a negligibly small volume) and all of the first substance in the lower inner barrel chamber 124 has been delivered to the lower outer barrel chamber 114 and mixed with the second substance.

Although the movement of inner barrel internal seal 126 causes the mixture of the first and second substances, in some instances additional agitation or mixing of the two substances may be advantageous or necessary. Additional mixing may be facilitated manually by the device user (e.g., by shaking or tapping the device to improve the mixing of the first and second substances), or by including mixing structures (e.g., ribs or vanes) in the drug delivery system 100. These may include a piezoelectric vibrating element, internal vanes or ribs in one or more of the second vacuum seal or lower outer barrel chamber 114, or other active or passive structures to create turbulence and/or promote mixing.

Delivery of the first substance into the lower outer barrel chamber increases the pressure within lower outer barrel chamber 114, causing outer barrel internal seal 116 to move toward the first end 101 of the outer barrel 110 (i.e., upward), as shown in FIG. 2. The final position of the outer barrel seal 116 within the outer barrel 110 may be selected based upon the starting pressures within the upper and lower outer barrel chambers 112, 114 and the volume of the first substance delivered from the lower inner barrel chamber 124 to the lower outer barrel chamber 114. It will be appreciated that because the volume within upper outer barrel chamber 112 decreases as the first substance is delivered into the lower outer barrel chamber 114, the pressure within the upper outer barrel chamber 112 will increase above its initial vacuum pressure in the position shown in FIG. 1, but may be selected so as to preserve a vacuum pressure sufficient to allow the injection of the mixed first and second substances into a desired target tissue of the patient, as illustrated in FIG. 3.

Once the mixed first and second substances are present in the lower outer barrel chamber 114, the drug delivery system 100 may be used to deliver the mixture to a target tissue of the patient. It will be appreciated that the user of the drug delivery system 100 must first insert the needle 109 into a desired target tissue of the patient. Once the needle 109 is inserted, delivery of the mixed first and second substances is accomplished by breaking a third vacuum seal 106. The third vacuum seal is provided at the upper end of outer barrel 110 and operates to seal a vacuum in the upper outer barrel chamber 112. As shown in FIGS. 1-3, third vacuum seal 106 comprises an annular or donut-shaped area. It is attached on its outer periphery to the outer wall of the outer barrel 110, and on its inner periphery to the wall defining the boundary between the inner barrel 120 and the outer barrel 110. Like the first and second vacuum seals 104, 105, the third vacuum seal 106 may comprise any of a variety of known seal types. Third vacuum seal 106 preferably comprises a seal that is easily broken by a user but is resistant to accidental breakage. In the embodiment of FIGS. 1-3, third vacuum seal 106 is a rupturable membrane seal.

When the third vacuum seal 106 is broken as shown in FIG. 3, entry of air into the upper outer barrel chamber 112 raises the vacuum pressure in the chamber 112 to atmospheric pressure and causes a downward force to be applied to outer barrel internal seal 116, moving the seal 116 toward the bottom of outer barrel 120. The mixed first and second substances in the lower outer barrel chamber 114 are injected through needle 109 into a desired target tissue of the patient. In some embodiments, including the embodiments of FIGS. 1-3, a one-way check valve 108 may be provided between the second end 103 of outer barrel 110 and needle 109. By selection of appropriate vacuum pressures within one or more of the upper and lower outer barrel chambers 112, 114, the force applied to outer barrel internal seal 116 when the third vacuum seal 106 is broken may be controlled to deliver the mixed first and second substances from the lower outer barrel chamber 114 at a desired delivery rate, or to ensure that viscous materials may be delivered to the lower outer barrel chamber 114 within a desired time period.

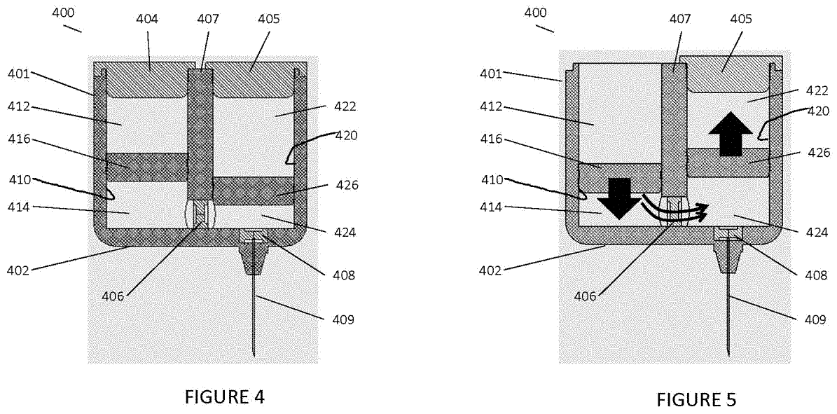

Another embodiment of a drug delivery system according to the prevent invention is illustrated FIGS. 4-7, with like numbers referring to like elements in each figure. FIG. 4 is a cross-sectional side view diagram of a drug delivery system 400. A plurality of chambers having vacuum pressures assist in the mixing of first and second desired substances within the device, and in the delivery of the mixed substances to a target tissue of a patient. The drug delivery system 400 of FIGS. 4-7 employs a parallel chamber design having a first barrel 410 and a second barrel 420, generally parallel with the first barrel 410, within a housing having a first or upper housing end 401 and a second or lower housing end 402. First and second barrels 410, 420 are separated by an interbarrel wall 407. A first barrel internal seal 416 separates the first barrel volume into an upper first barrel chamber 412 and a lower first barrel chamber 414. Second barrel 420 includes a second barrel internal seal 426 that separates the second barrel volume into an upper second barrel chamber 422 and a lower second barrel chamber 424. In various embodiments, the first barrel internal seal 416 and the second barrel internal seal 426 may be selected from an 0-ring and a movable elastomeric plug.

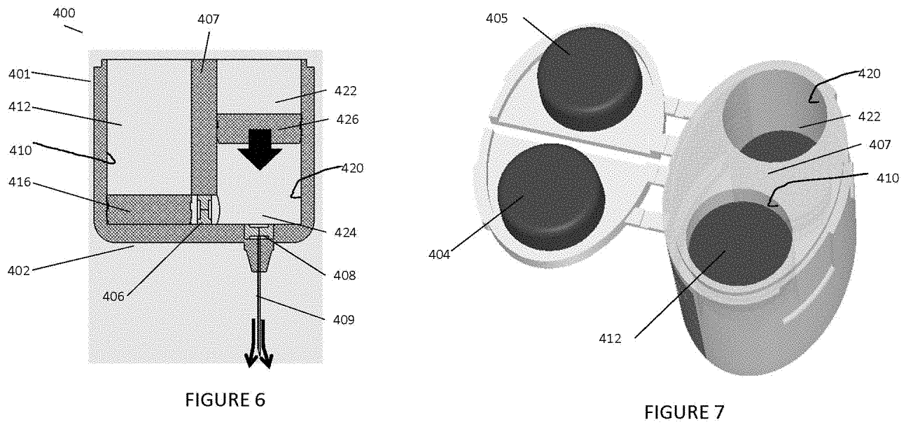

A first substance (not shown), such as a carrier for an active agent, is provided in the lower first barrel chamber 414. A desired vacuum pressure is provided in lower first barrel chamber 414, which is defined by the lower surface of first barrel internal seal 416 and the lower end of first barrel 410. A first vacuum seal 404 is provided at the upper end of first barrel 410 to seal a vacuum within the upper first barrel chamber 412, defined by the first vacuum seal 404 and the upper surface of first barrel internal seal 416. The vacuum pressure in upper and lower first barrel chambers 412, 414 may be selected so as to control the force applied to the upper surface of first barrel internal seal 416 when the first vacuum seal 404 is ruptured. A greater force may be selected to deliver the first substance from the lower first barrel chamber 414 to the lower second barrel chamber 424 faster, or to ensure that viscous materials may be efficiently delivered to the lower second barrel chamber 424. First vacuum seal 404 is coupled to the housing first end 401 and the interbarrel wall 407. In some embodiments (see FIG. 7), first barrel 410 may comprise a circular cross section, and first vacuum seal 404 may comprise a stopper to sealingly engage the housing first end and the interbarrel wall 407. It should be appreciated, however, that first vacuum seal 404 may comprise any of a variety of known seal types, e.g., a resilient stopper, a rupturable or puncturable membrane, etc. In preferred embodiments, first vacuum seal 404 is a seal that is easily broken by a user but is resistant to accidental breakage or rupture. In the embodiment of FIGS. 4-7, first vacuum seal 404 is a removable stopper.

FIG. 5 illustrates how first vacuum seal 404 may be ruptured to cause the first substance to be delivered from the lower first barrel chamber 414 to the lower second barrel chamber 424 for mixing with a second substance present in the lower second barrel chamber. When the first vacuum seal 404 is broken, entry of air into the upper inner first barrel chamber 412 raises the pressure in the chamber to atmospheric pressure and causes a net downward force to be applied to first barrel internal seal 416, moving the seal 416 toward the bottom of first barrel 410, as shown by the downward arrow in FIG. 5. The first substance in the lower first barrel chamber 414 is driven through an interbarrel one-way valve 406 in the interbarrel wall 407 separating the first and second barrels 410, 420. Interbarrel one-way valve only permits flow of the first substance from the from the lower first barrel chamber 414 to the lower second barrel camber 424, as indicated by the directional arrows through interbarrel one-way valve 406 in FIG. 5. When the first barrel internal seal 416 reaches the bottom of the first barrel 410, the volume of lower first barrel chamber 414 is reduced to zero, and all of the first substance is mixed with the second substance in lower second barrel chamber 424. It will be appreciated that the interbarrel one-way valve 406 may be replaced by other flow control elements or seals that permit only one-way flow of the first substance from the lower first barrel chamber 414 to the lower second barrel chamber 424.

Although the movement of first barrel internal seal 416 causes the mixture of the first and second substances, in some instances additional agitation or mixing of the two substances may be facilitated manually by the device user or by including mixing structures as described in connection with FIGS. 1-3.

Delivery of the first substance into the lower second barrel chamber 424 increases the pressure within the chamber, causing second barrel internal seal 426 to move toward the housing first end 401 of the second barrel 420 (i.e., upward), as indicated by the upward directional arrow in FIG. 5. The final position of the second barrel seal 426 within the second barrel 420 may be selected based upon the starting pressures within the upper and lower first barrel chambers 412, 414 and the volume of the first substance delivered from the lower first barrel chamber 414 to the lower second barrel chamber 424. It will be appreciated that because the volume within upper second barrel chamber 422 decreases as the first substance is delivered into the lower second barrel chamber 424, the pressure within the upper second barrel chamber 422 will increase above its initial vacuum pressure in the position shown in FIG. 4, but may be selected so as to preserve a vacuum pressure sufficient to allow the injection of the mixed first and second substances into a desired target tissue of the patient, as illustrated in FIG. 6.

Once the mixed first and second substances are present in the lower second barrel chamber 424, the drug delivery system 400 may be used to deliver the mixture to a target tissue of the patient. It will be appreciated that the user of the drug delivery system 400 must first insert the needle 409 into a desired target tissue of the patient. Once the needle 409 is inserted, delivery of the mixed first and second substances is accomplished by breaking a second vacuum seal 405 located at the upper end of second barrel 420. Second vacuum seal 405 operates to seal a vacuum pressure (e.g., 0.5 atm) in the upper second barrel chamber 422. In some embodiments, the second barrel 420 may comprise a circular cross section and second vacuum seal 405 may comprise a stopper (FIGS. 4, 5 and 7) to sealingly engage the housing first end and the interbarrel wall 407. It should be appreciated, however, that first vacuum seal 404 may comprise any of a variety of known seal types, e.g., a resilient stopper, a rupturable or puncturable membrane, etc. In preferred embodiments, first vacuum seal 404 is a seal that is easily broken by a user but is resistant to accidental breakage or rupture.

Referring again to FIG. 6, when the second vacuum seal 106 is broken, entry of air into the upper second barrel chamber 422 raises the vacuum pressure in the chamber 422 to atmospheric pressure and causes a downward force to be applied to second barrel internal seal 426 as indicated by the downward arrow on the seal 426. The downward force moves the second barrel internal seal 426 toward the bottom of second barrel 420. The mixed first and second substances in the lower second barrel chamber 424 are injected through needle 409 into a desired target tissue of the patient. In some embodiments, including the embodiments of FIGS. 4-6, a needle one-way valve 408 may be provided in the second housing end at the bottom of lower second barrel chamber 424. In other embodiments, needle one-way valve 408 may be omitted. By selection of appropriate vacuum pressures within one or more of the upper and lower second barrel chambers 422, 424, the force applied to second barrel internal seal 426 when the second vacuum seal 405 is broken may be controlled to deliver the mixed first and second substances from the lower second barrel chamber 424 at a desired delivery rate, or to ensure that viscous materials may be delivered to the target body tissue of the patient within a desired time period.

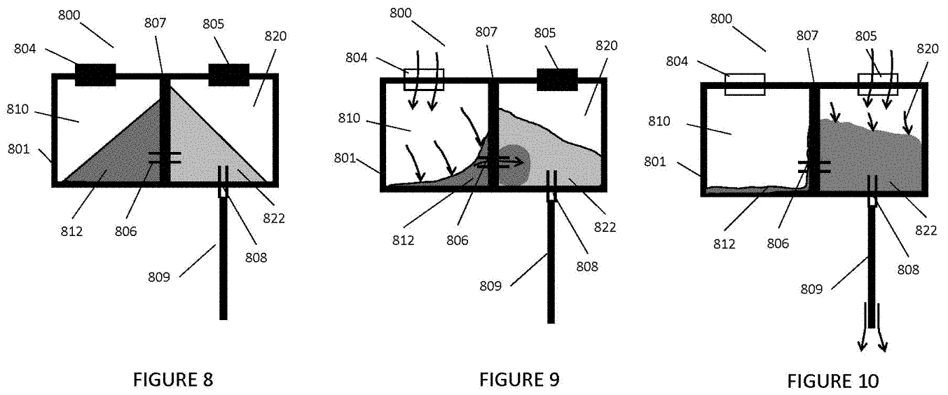

Another embodiment of a drug delivery system according to the prevent invention is illustrated FIGS. 8-10, in which like numbers are used to refer to like elements in each of the figures. FIG. 8 is a schematic cross-sectional view of a drug delivery system 800, in which plurality of chambers having vacuum pressures are used to assist in the mixing of first and second desired substances within the system, and in the delivery of the mixed substances from the system to a target tissue of a patient. The drug delivery system 800 of FIGS. 8-10 includes a first chamber 810 and a second chamber 820 that are connected at least in part by an interchamber wall 807. An interchamber one-way valve 806 is provided in a selected location of the interchamber wall. The system further includes a housing 801 defining the outer periphery of at least a portion of the first chamber 810 and/or the second chamber 820. The housing 801 may be made of relatively hard materials capable of withstanding vacuum pressure used in the system without buckling or collapsing. The housing 801 may be rigid, semi-rigid or flexible so long as it is capable of withstanding the vacuum pressures used in the system, which may range from nearly absolute vacuum to pressures just below atmospheric pressure.

A first substance is provided inside first chamber 810 in a flexible first substance container 812, which may comprise any of a variety of containers that are flexible, collapsible, or squeezable. In various embodiments, the flexible first substance container 812 may comprise a flexible bag or ball and may comprise a flexible membrane in a desired shape suitable for providing a reservoir for the first substance, depicted in grayscale in FIG. 8 within flexible first substance container 812. First chamber 810 and flexible first substance container 812 may initially be provided at a desired vacuum pressure (e.g., 0.35 atm). A first vacuum seal 804 is provided to seal a vacuum within the first chamber 810. First vacuum seal 804 may comprise any of a number of sealing mechanisms, such as a removable stopper, a releasable valve, or a membrane seal that may be ruptured or punctured. Preferably, first vacuum seal 804 is easily broken or opened by a user but is resistant to accidental opening, breakage, or rupture. In the embodiment of FIGS. 8-10, first vacuum seal 804 is a releasable valve.

A second substance, such as a drug, may be provided in a flexible second substance container 822 inside second chamber 820. Like the flexible first substance container 812, the flexible second substance container 822 may comprise any of a variety of flexible, collapsible, or squeezable containers in a desired shape suitable for providing a reservoir for the second substance, which is also depicted in grayscale in FIG. 8 within flexible second substance container 822. A second vacuum seal 805 is provided to seal a vacuum within the second chamber 820 and may comprise any of a number of sealing mechanisms, similar to first vacuum seal 804.

The initial vacuum pressure within first chamber 810 may be selected to control the force applied to the flexible first substance container 812 when the first vacuum seal 804 is opened or ruptured. A greater vacuum (and correspondingly greater force applied to the flexible first substance container 812 upon release of the vacuum) may be selected to deliver the first substance from the flexible first substance container 812 in the first chamber 810 to mix with the second substance in the flexible second substance container 822 in the second chamber 820 via the interchamber one-way valve 806. Greater vacuum levels will cause the first substance to mix with the second substance more rapidly and may be selected to ensure that viscous first substances such as a solvent for a drug may be efficiently delivered from the flexible first substance container 812 to the flexible second substance container 822.

FIG. 9 illustrates how first vacuum seal 804 may be opened to cause the first substance to be delivered from the flexible first substance container 812 to the flexible second substance container 822 for mixing with the second substance. When the first vacuum seal 804 is opened, entry of air into the first chamber 810 (indicated by the arrows in vacuum seal 804 and first container 810) raises the pressure in the chamber to atmospheric pressure and causes the flexible first substance container 812 to collapse, driving the first substance through the interchamber one-way valve 806 in the interchamber wall 807 and into the flexible second substance container 822. Interchamber one-way valve 806 only permits flow of the first substance from the from flexible first substance container 812 into the flexible second substance container and prohibits flow of the second substance from the flexible second substance container 822 into the flexible first substance container 812. It will be appreciated that the interchamber one-way valve 806 may be replaced by other flow control elements or seals that permit only one-way flow of the first substance from the flexible first substance container 812 to the flexible second substance container 822.

Although the collapse of the flexible first substance container 812 causes the mixture of the first and second substances, in some instances additional agitation or mixing of the two substances may be facilitated manually by the device user or by including mixing structures inside the second chamber 820 or the flexible second substance container 822.

Delivery of the first substance into the flexible second substance container 822 increases the pressure within the container 822, causing its volume to increase and also increasing the pressure within second chamber 820 (FIG. 9). The starting pressure inside second chamber 820 may be selected so as to preserve a vacuum pressure within the chamber 820 sufficient to allow the injection of the mixed first and second substances into a desired target tissue of the patient, as illustrated in FIG. 10.

Once the mixed first and second substances are present in the flexible second substance container 822, the drug delivery system 800 may be used to deliver the mixture to a target tissue of the patient (FIG. 10). It will be appreciated that the user of the drug delivery system 800 must first insert the needle 809 into a desired target tissue of the patient. Once the needle 809 is inserted, delivery of the mixed first and second substances is accomplished by opening a second vacuum seal 805 located on the second chamber 820.

Referring again to FIG. 10, when the second vacuum seal 805 is opened, entry of air into the second chamber 820 raises the vacuum pressure in the chamber 820 to atmospheric pressure and causes a force to be applied to the mixture of the first and second substances within flexible second substance container 822, collapsing it and driving the mixture through a needle one way valve 808 and needle 809 and into the target tissue of the patient, as indicated by the downward arrows in second vacuum seal 805 and second chamber 820 in FIG. 10. In some embodiments, needle one-way valve 808 may be omitted. By selection of appropriate vacuum pressures within one or more of the first and second chambers 810, 820, the force applied to flexible first and second substance containers 812, 822 may be controlled to mix and/or deliver the first and second substances to the target body tissue of the patient.

What is claimed:

1. A system for delivery of at least one of a first substance and a second substance to a target body tissue of a patient, comprising:
a first chamber having a first vacuum pressure and housing a first substance for delivery to a target body tissue of a patient;
a second chamber having a second vacuum pressure and housing a second substance for delivery to a target body tissue of a patient;
a first flow control element coupling said first chamber to said second chamber, wherein the first flow control element permits fluid communication from said first chamber to said second chamber;
a first vacuum seal, wherein the first vacuum seal, when opened, exposes the first chamber to atmospheric pressure and causes the first substance to be delivered from the first chamber to the second chamber and mixed with the second substance; and
a second vacuum seal, wherein the second vacuum seal, when opened, exposes the second chamber to atmospheric pressure and causes the contents of the second chamber to be delivered to the target body tissue of the patient.

2. The system of claim 1, wherein the first flow control element comprises at least one of a one-way check valve, a rupturable vacuum seal, and a rupturable membrane.

3. The system of claim 1, wherein the first substance and the second substance are selected from a pharmacological agent, a diluent for the pharmacological agent, and a carrier for the pharmacological agent.

4. The system of claim 1, further comprising a needle coupled to the second chamber, wherein the second vacuum seal, when opened, exposes the second chamber to atmospheric pressure and causes the contents of the second chamber to be delivered to the target body tissue of the patient through the needle.

5. The system of claim 4, further comprising a second flow control element coupling the second chamber and the needle, wherein the second flow control element is selected from a rupturable seal and a one-way check valve.

6. The system of claim 1, wherein the first vacuum pressure is different from the second vacuum pressure, and wherein the first vacuum pressure and the second vacuum pressure are selected from a pressure within the range of 0.01 atm to 0.99 atm.

7. The system of claim 6, wherein the first vacuum pressure and the second vacuum pressure are selected from a pressure within the range of 0.25 at to 0.75 atm.

8. The system of claim 1, further comprising a housing defining at least a portion of said first chamber and said second chamber.

9. The system of claim 1, wherein at least one of said first and said second chambers is collapsible.

10. The system of claim 1, further comprising:
a third chamber having a third vacuum pressure and housing a third substance for delivery to a target body tissue of a patient;
a second flow control element coupling said third chamber to said second chamber, wherein the second flow control element permits fluid communication from said third chamber to said second chamber; and
a third vacuum seal, wherein the third vacuum seal, when opened, exposes the third chamber to atmospheric pressure and causes the second substance to be delivered from the third chamber to the second chamber and mixed with the second substance.

11. A method for delivery of at least one of a first substance and a second substance to a target body tissue of a patient, comprising:
providing a first chamber having a first vacuum pressure and housing a first substance for delivery to a target tissue of a patient;
providing a second chamber having a second vacuum pressure and housing a second substance for delivery to a target tissue of a patient;
providing a first flow control element coupling said first chamber to said second chamber, wherein the flow control element permits fluid communication from said first chamber to said second chamber;
providing a first vacuum seal that, when opened, exposes the first chamber to atmospheric pressure and causes the first substance to be delivered from the first chamber to the second chamber and mixed with the second substance;
providing a second vacuum seal that, when opened, exposes the second chamber to atmospheric pressure and causes the contents of the second chamber to be delivered to the target body tissue of the patient;
opening said first vacuum seal; and
opening said second vacuum seal.

12. A system for delivery of at least one of a first substance and a second substance to a target body tissue of a patient, comprising:
a first barrel comprising:
an upper first barrel chamber having a first vacuum pressure;
a lower first barrel chamber having a second vacuum pressure and housing a first substance for delivery to the target body tissue of the patient; and
a movable first barrel internal seal separating the upper first barrel chamber from the lower first barrel chamber;
a second barrel comprising:
an upper second barrel chamber having a third vacuum pressure and comprising a second vacuum seal that, when opened, exposes the upper second barrel chamber to atmospheric pressure;
a lower second barrel chamber having a fourth vacuum pressure and housing a second substance for delivery to the target body tissue of the patient; and
a movable second barrel internal seal separating the upper first barrel chamber from the lower first barrel chamber;
a first flow control element coupling said first chamber to said second chamber, wherein the first flow control element permits fluid communication from said first chamber to said second chamber;
a first vacuum seal coupled to the upper first barrel chamber that, when opened, exposes the upper first barrel chamber to atmospheric pressure and causes the first substance to be delivered from the lower first barrel chamber to the lower second barrel chamber and mixed with the second substance; and
a second vacuum seal coupled to the upper second barrel chamber that, when opened, exposes the upper second barrel chamber to atmospheric pressure and causes the contents of the lower second barrel chamber to be delivered to the target body tissue of the patient.

13. The system of claim 12, wherein the first flow control element comprises at least one of a one-way check valve, a rupturable vacuum seal, and a rupturable membrane.

14. The system of claim 12, wherein the first substance and the second substance are selected from a pharmacological agent, a diluent for the pharmacological agent, and a carrier for the pharmacological agent.

15. The system of claim 12, further comprising a needle coupled to the lower second barrel chamber, wherein the second vacuum seal, when opened, exposes the upper second barrel chamber to atmospheric pressure and causes the contents of the lower second barrel chamber to be delivered to the target body tissue of the patient through the needle.

16. The system of claim 15, further comprising a second flow control element coupling the lower second barrel chamber and the needle, wherein the second flow control element is selected from a rupturable seal and a one-way check valve.

17. The system of claim 12, wherein at least one of the first vacuum pressure, the second vacuum pressure, the third vacuum pressure, and the fourth vacuum pressure is different from the others of said first vacuum pressure, the second vacuum pressure, the third vacuum pressure, and the fourth vacuum pressure, and wherein the first vacuum pressure, the second vacuum pressure, the third vacuum pressure, and the fourth vacuum pressure are selected from a pressure within the range of 0.01 atm to 0.99 atm.

18. The system of claim 17, wherein first vacuum pressure, the second vacuum pressure, the third vacuum pressure, and the fourth vacuum pressure are selected from a pressure within the range of 0.25 at to 0.75 atm.

19. The system of claim 12, further comprising a housing defining at least a portion of said first barrel and said second barrel.

20. The system of claim 12, wherein the lower first barrel chamber and the lower second barrel chambers are collapsible.
